(19) United States
(12) Patent Application Publication
(10) Pub. No.: US 2015/0196360 A1
(43) Pub. Date: Jul. 16, 2015

(54) Medical Device for Revascularization of Vascular Occlusion and Method for Using Same

(71) Applicants: James Aaron Grantham, Overland Park, KS (US); Praneet Kumar Sharma, Shawnee, KS (US)

(72) Inventors: James Aaron Grantham, Overland Park, KS (US); Praneet Kumar Sharma, Shawnee, KS (US)

(21) Appl. No.: 14/575,977

(22) Filed: Dec. 18, 2014

Related U.S. Application Data
(60) Provisional application No. 61/917,916, filed on Dec. 18, 2013.

(51) Int. Cl. A61B 18/24; A61M 25/09; A61B 8/12; A61B 5/00; A61B 18/14; A61M 25/01

(52) U.S. Cl. CPC A61B 18/245; A61B 18/1492; A61M 25/0194; A61B 8/12; A61B 5/0066; A61M 25/09; A61B 2018/0041

(57) ABSTRACT

An improved medical device for revascularization of vascular occlusion and method for using same. The improved device includes a first catheter with a central lumen or guide wire capable of creating a channel within the CTO, and having a magnetic connection tip. One or more magnets or magnetic alignment elements are secured within or on the catheter connection tip to provide the desired positioning or straight line alignment of the catheter tip with respect to a second guide wire. The second guide wire includes a magnetic connection tip, with one or more magnets or magnetic alignment elements secured within or on the wire connection tip. The magnets or magnetic alignment elements on each connection tip are arranged such that opposite polarity magnets are used to attract the magnetic tips and their respective wire or catheter into the occlusion or subintimal space and into a close proximity position and alignment within the vessel to enable the use of mechanical elements, wires, energy sources, laser sources, or combinations of thereof, to assist in crossing the occlusion by creating a channel.

### CROSS REFERENCE TO RELATED APPLICATION

The present application claims priority from U.S. Provisional Patent Application Ser. No. 61/917,916 filed Dec. 18, 2013, which is incorporated herein by reference.

### FIELD OF INVENTION

The present application relates to facilitating revascularization of vascular occlusion in human blood vessels, and to a device and method for forming a connection across occluded blood vessels by improving position, orientation and connection between devices used to revascularize an occluded segment of the vessel.

### BACKGROUND

Numerous solutions have been offered in the field of interventional cardiology and radiology to address the treatment of blood vessels in the human body which are seriously stenosed or have been occluded. Some of these vessels may have a chronic total occlusion (CTO), in which a vessel is completely occluded and no longer enables the passage of blood. Such a vessel may lie in the blood vessels of the heart (coronary arteries) or in the peripheral vasculature supplying the extremities or other vital organ systems. It is known in the field of interventional cardiology that these blockages may cause symptoms due to lack of sufficient blood supply to the structures distal to the occlusion, such as, the heart tissue. In the heart tissue, a CTO is associated with angina pectoris and poor quality of life. Opening these vessels by use of existing technologies, such as balloon angioplasty and stenting or percutaneous coronary interventions may improve symptoms and quality of life.

Various procedures are performed in specialized interventional laboratories in contemporary medical practices (as opposed to full surgical procedures) to open a CTO. However, there are several limitations to existing procedures and technologies. First, such techniques are very difficult to master, such that the results are not uniform and success rates are low (50-70%) even when performed by very experienced medical practitioners. A main difficulty lies in advancing a guide wire through a CTO so that other devices required to complete the procedure can be delivered to open the occlusion. Such devices include percutaneous transluminal coronary angioplasty (PTCA) balloon catheter, a stent, a laser, and an atherectomy device among others currently available or in development. Traditionally, a guide wire is advanced in the blocked vessel towards the blockage and an attempt is made to advance the guide wire through the blockage. This is called an antegrade approach. Various techniques have been developed which employ specific wires and catheters to pass through the CTO in order to make a connection through the blockage to devices positioned on the other side of the blockage. Examples of such technologies include an assortment of specialty guide wires such as the Asahi Confianza PRO, by Abbott Vascular, Inc. of Santa Clara, Calif., the Wildcat device, manufactured by Avinger of Redwood City, Calif., and devices disclosed in U.S. Pat. No. 8,911,534 to Katoh et al. Such techniques focus on passing through the true lumen of the vessel (the intraluminal techniques) while navigating complex stenosis.

In many severe blockages, including CTO's, the antegrade approach is unsuccessful in crossing the occlusion. Consequently, an adjunctive technique to improve procedural success rates and reduce procedural time has been developed. This additional procedure involves using existing natural connections provided by body systems, such as via marginal branches that connect major vessels. These natural pathways enable the occlusion site or CTO to be approached with devices from the opposite direction, which is referred to as the retrograde approach. The main difficulty using this technique is making a connection between devices on one end of the occluded vessel to the other end. Several devices have been developed or are under development which propose methods of forming connection between true lumen from the proximal to the distal true lumen of a blood vessel by specialized devices such as wires, catheters delivering force or energy such as radio frequency energy. However, almost one third of all coronary CTO lesions are longer than 20 mm in length. Therefore, if force or energy is delivered across such long lesions, it may deliver this energy towards the adventitia (or outer layer) of the blood vessel and perhaps cause harmful effects, such as perforation of the blood vessel or other complications or failure of the procedure. Other techniques for passing the CTO employ the use of subintimal space or planes outside of the vessel lumen (through the vessel wall) to traverse the stenosis and regain entry to the true lumen (subintimal techniques). Devices employed in this technique include guide wires and catheters, used alone or in tandem, to adjust and enhance device tip attributes deemed useful in crossing or passing through the occlusion. An example of such a device includes a highly flexible guide wire, an Asahi Sion, by ASAHI INTECC CO., LTD of Aichi, Japan, which is delivered through a support catheter such as a FineCross MG coronary micro-guide catheter by Terumo Corporation of Tokyo, Japan. Numerous and various devices of the types illustrated are used interchangeably by medical practitioners to cross difficult vessel occlusions. Once a connection is made between the devices delivered from opposite ends of the occlusion, either through or around the occlusion, the devices (such as a wire) are either extended or externalized so that technical equipment, such as balloon angioplasty equipment and stents, can be passed and used to open the blockage.

The main limiting step with the retrograde technique in this process is making the connection between the devices through the CTO between the proximal and distal ends of the occlusion inside the vessel. Although these novel techniques and devices have been developed, they have significant limitations, such as the need for practitioner expertise, difficulty in learning and teaching the techniques (unlike other interventional techniques), thus limiting widespread use, requiring long procedural time, the need for multiple devices such as wires which may fail and thus be discarded, adding to the cost of the procedure, and most importantly, high rates of complications which may result from inadvertently perforating a vessel. There is a need to address these limitations by providing a safe, effective and efficient way to overcome these problems.

### SUMMARY OF THE INVENTION

The present device provides a number of improved features over prior devices and methods. The device provides an easier and simpler way of connecting the devices used during a retrograde technique, on opposite sides of the CTO or other defect, so that they are positioned in the closest proximity in order to enable easy connection using magnets positioned on the tips of both of the devices. The device includes a first catheter or guide wire, having a magnetic connection tip and provides for delivery of energy at the tip. Energy is delivered or received via an electrode mounted near the tip. The first catheter or guide wire is advanced from the antegrade direction toward the CTO. A second guide wire or catheter, is also provided having a magnetic connection tip and providing for delivery or receiving of energy at the tip. The second guide wire or catheter is advanced toward the occlusion site via an adjacent vessel to enable an approach to the distal end of the occlusion, opposite from the first guide wire or catheter. It is understood that it is desirable to minimize the distance between the electrodes mounted on the first and second guide wire or catheter to reduce the likelihood of misdirecting energy. The wire and catheter are positioned with their tips in close proximity but separated by intervening vascular tissue, such that one device is positioned within the true lumen and the other inside the wall of the vessel, the latter position referred to as subintimal. The tips of each device may be proximal or distal to the CTO. One tip may be within the arterial occlusion or plaque and the other connection tip adjacent to it within the subintimal space. Providing increased connection locations offers the medical practitioner flexibility in positioning each device in order to make the connection.

During use of the device in a procedure to recanalize a CTO, the individual components of the device are inserted inside the body by methods which are well known and practiced by experts in this field, for example, the controlled antegrade and retrograde tracking (CART) technique or a reverse CART technique, which utilize one component, typically the catheter, to be advanced in antegrade manner to the CTO proximal cap, while the other second component, the guide wire, is advanced in retrograde manner towards the distal end of the CTO. The wire and catheter are guided using the desired techniques to positions within the vessel on either side of the occlusion, and the wire is then guided into the occlusion or adjacent the occlusion within the subintimal space, until the magnetic tip of the wire is positioned within the closest proximity to the magnetic tip of the catheter. Using the magnetic force, the tips of the devices will move into a desired aligned position, based upon placement of the magnets on the respective instrument tips, and will form and lie in a straight line or a nearly connected straight line. The intervening tissue separating the wire and the catheter may then be more easily breached by inserting into the occlusion a mechanical element, such as a hard tip wire, blade, needle or the like, or other such techniques that are known to medical practitioners in this field. Alternatively, energy such as electromagnetic force, radio frequency or a laser may be used to form a channel, breach the tissue and create a connection. The tips in this aligned and close proximity position, require less energy and heat to be applied between the devices to form the channel, thereby reducing the risks associated with the use of high levels of energy and heat to the vessel. A wire may then be threaded through the channel to move the wire into a central lumen of the catheter, whereupon a complete physical loop connection is created. The wire may be substituted for other devices such as balloon catheters, stents or other appropriate devices, that can be passed over the wires to open the occlusion for revascularization, as is known in the field.

### DESCRIPTION OF THE DRAWINGS

FIG. 1 is a schematic illustration of a cardiovascular system having a wire and a catheter of the device of the present application depicted extending through the vasculature using a conventional reverse CART technique to place the devices at locations on the proximal and distal sides of a chronic total occlusion within a vessel.

FIG. 2a is a schematic close-up view of the occlusion shown in FIG. 1, with the magnetic tip catheter and the magnetic tip wire shown within the occlusion, and somewhat within the subintimal space, in aligned position under attraction of the opposite polarity tip magnets.

FIG. 2b is a schematic close-up view of the occlusion shown in FIG. 2, but with the magnetic tip catheter and the magnetic tip wire shown connected within the occlusion to form a straight line, and somewhat within the subintimal space, and forming a channel through the occlusion.

FIG. 3a illustrates a schematic side view of the magnetic tip of the catheter instrument, and FIG. 3b illustrates a schematic end view of the tip of the catheter with a lumen for use in supplying additional devices through the catheter, and magnetic tip.

FIG. 4a illustrates a schematic side view of the magnetic tip of the wire instrument, and FIG. 4b illustrates a schematic end view of the tip of the wire with a central core, and a surrounding magnetic tip.

FIG. 5a is a schematic illustration of a side view of an alternate embodiment of a wire instrument having a U-shaped orientation for reentry into the vessel lumen and showing a magnetic tip supporting an arrangement of multiple magnets surrounding a central core opening, and FIG. 5b is a schematic cross-sectional view of the center core of the wire, and FIG. 5c is a schematic close-up view of the magnetic tip of the alternate wire embodiment.

FIG. 6a is a schematic illustration showing use of a magnetic tip of a catheter instrument extending into the CTO and attracted to a straight line position with the magnetic tip of the wire.

FIG. 6b is a schematic illustration showing use of a magnetic tip of a wire instrument extending into the CTO and attracted to a straight line position the magnetic tip of the catheter.

FIG. 6c is a schematic illustration showing use of a magnetic tip of a wire instrument extending into the subintimal space of a vessel adjacent a CTO.

FIG. 6d is a schematic illustration of FIG. 6c, but with the wire instrument in closer proximity to the magnetic tip of the catheter and able to more easily create a straight line connection between magnetically attracted wire and catheter for revascularization of the vessel.

FIG. 7 is schematic view of a heart with an atrial septal defect showing use of a guide with a magnetic tip approaching the defect from the left auricle from the proximal side, and a magnetic guide located within the right auricle of the heart being used to guide the magnetic catheter toward the defect.

FIG. 8a is a schematic illustration of a cardiovascular system having a wire and a catheter of the device depicted extending through the vasculature to place the devices at locations on the proximal and distal sides of a chronic total occlusion within a vessel.

FIG. 8b is a schematic close-up view of the occlusion shown in FIG. 8a, with the magnetic tip catheter shown adjacent the occlusion, and a non-activated electromagnetic coil embedded within the wire which is within the subintimal space.

FIG. 8c is a schematic close-up view of the occlusion shown in FIGS. 8a and 8b, but with the electromagnetic coil within the wire shown activated to magnetically attract to the magnetic tip of the catheter to form a connection.

### DETAILED DESCRIPTION

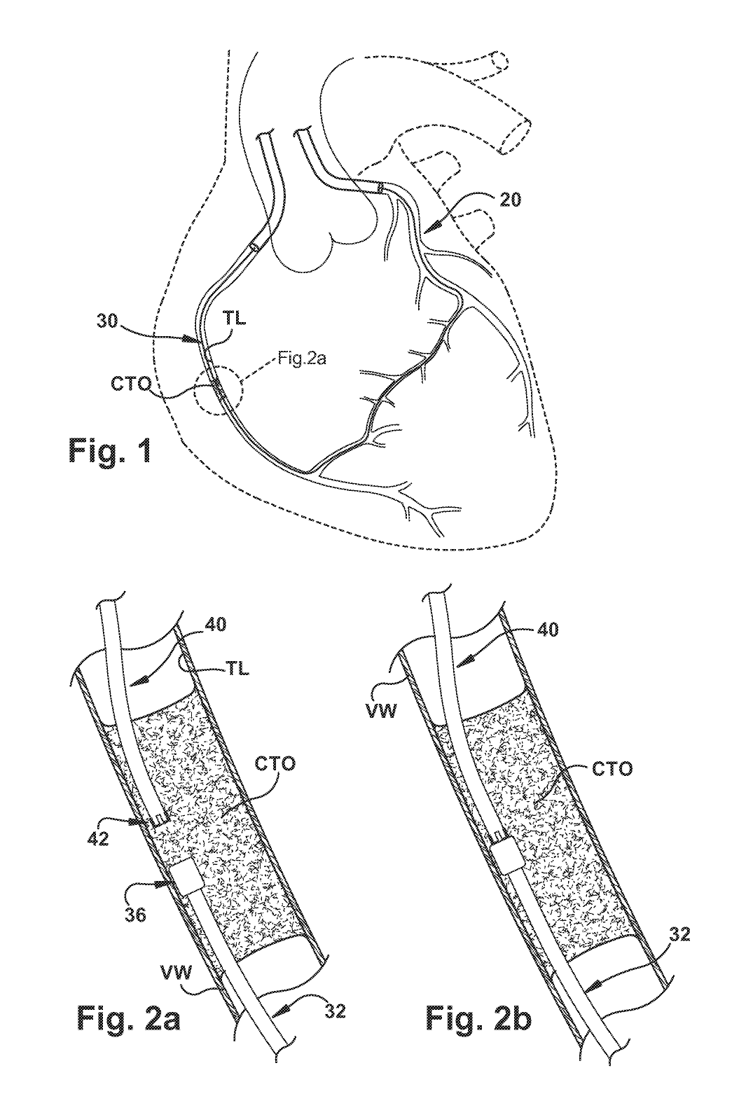

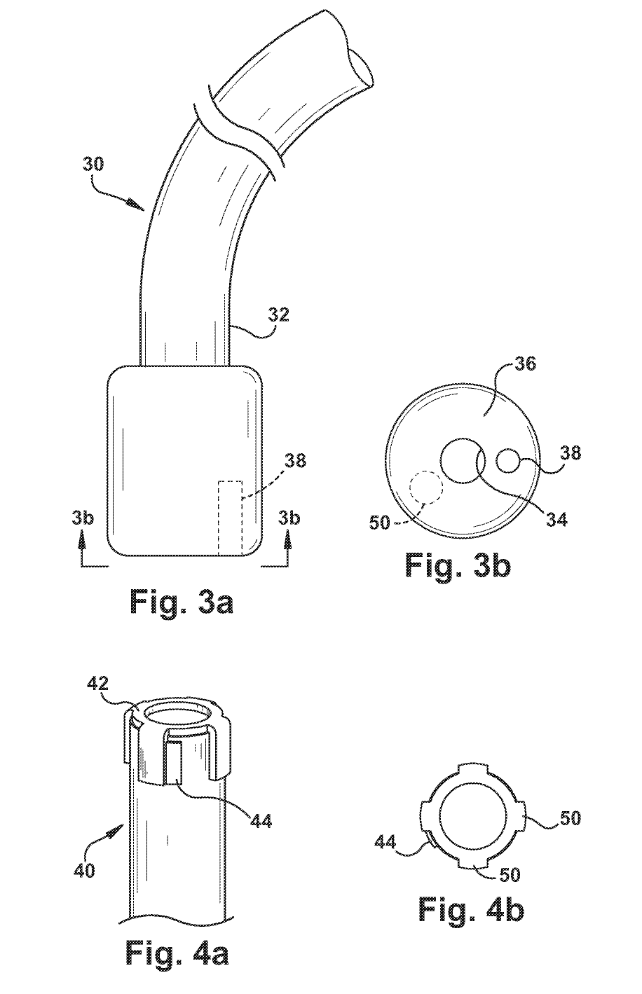

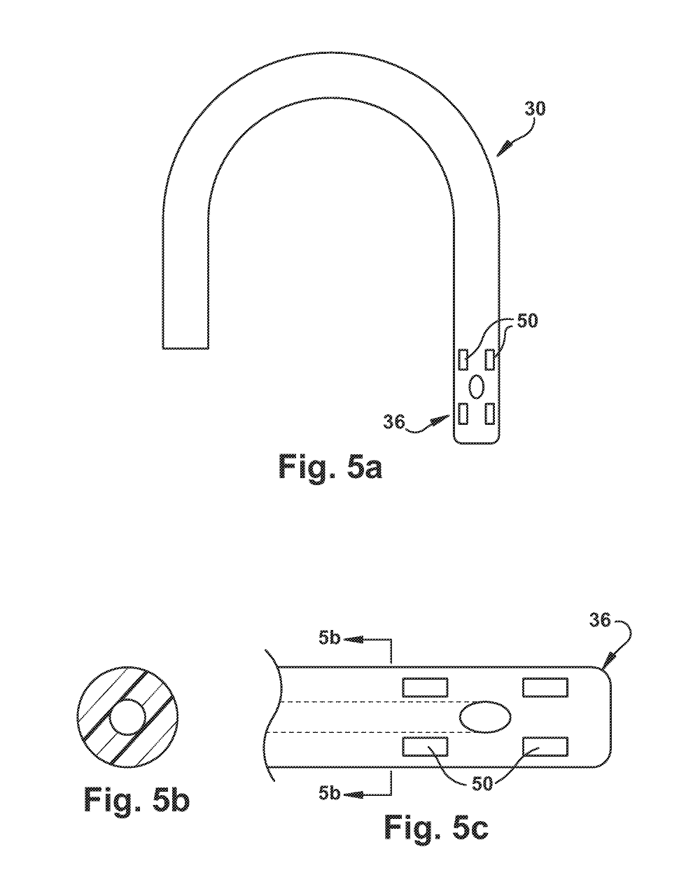

The present application provides an improved medical device 30, system and method for revascularization of a complete total occlusion CTO within a vessel V of the type schematically illustrated in the human vascular system 20 of FIG. 1. The device 30 includes a first guide 32, which may be a catheter including a central lumen 34 capable of facilitating passage of another catheter or guide wire, or a guide wire, and having a magnetic connection tip 36 to provide for delivery of energy at the tip. In the embodiment of FIGS. 3a and 3b, radiofrequency (RF) energy may be delivered or received via an electrode 38 mounted near the tip 36. As shown in FIG. 1, the first catheter or guide wire 32 is advanced from the antegrade direction to the CTO proximal cap or end of the CTO. One or more magnets or magnetic alignment elements, shown in FIGS. 3a, 3b and 5a, 5c, are secured within or on the catheter connection tip to provide the desired positioning or alignment of the catheter tip with respect to a second guide 40, which may be a guide wire or catheter, also having a magnetic connection tip 42 and providing for delivery or receiving of energy at the tip.

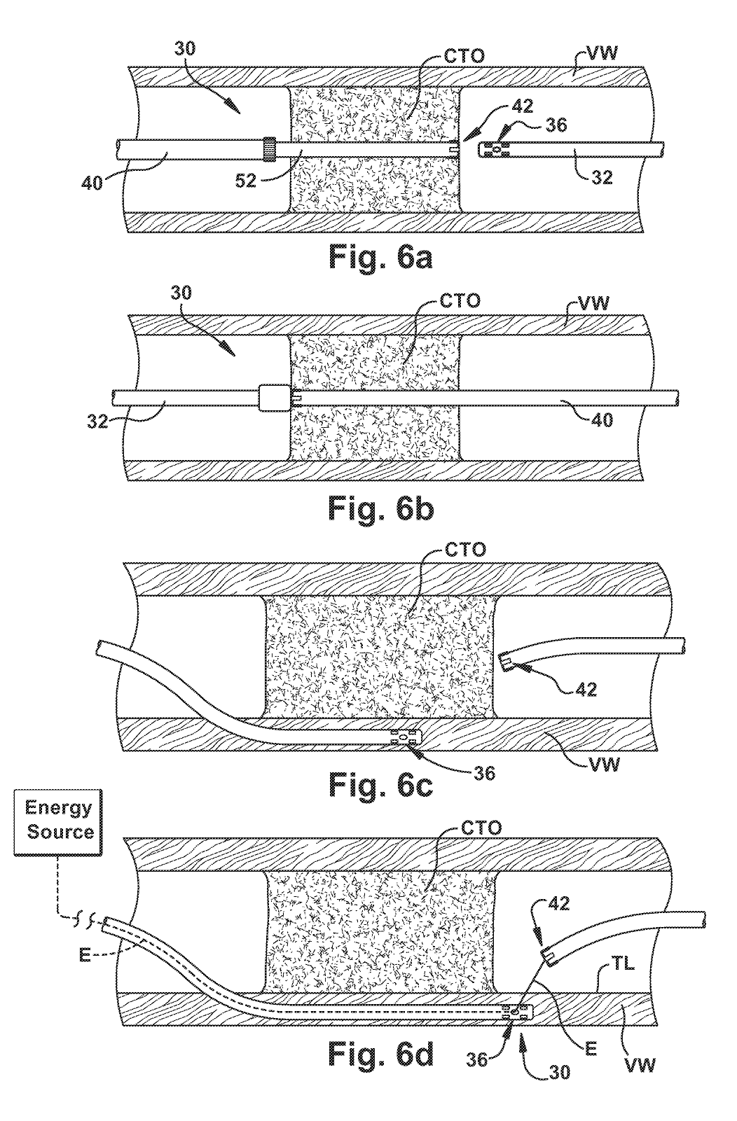

The second guide wire or catheter 40 is advanced to the occlusion site CTO via an adjacent vessel to enable an approach to the distal end of the occlusion, opposite from the first guide wire or catheter 32, as shown in FIGS. 1, 2a and 2b. Each of the guide wires or catheters 32, 40 include a magnetic connection tip, with one or more magnets or magnetic alignment elements secured within or on the connection tip. The magnets or magnetic alignment elements 50 on each connection tip 36, 42 are arranged such that opposite polarity magnets 50 are used to attract the magnetic tips 36, 42 and their respective guide wire or catheter 32, 40 to the desired relative position and alignment within the vessel. It is understood that it is desirable to minimize the distance between any electrodes mounted on the first and second guide wire or catheter 32, 40 to reduce the likelihood of misdirecting energy. It is also understood, and shown in FIGS. 2a, 6a and 6c, that the wire and catheter 32, 40 are positioned with their tips 36, 42 in close proximity but separated by intervening vascular tissue. In the embodiment shown in FIGS. 2a, 2b and 6c, 6d, one device 32 or 40 is positioned within the true lumen TL of the vessel and the other device 40 or 32 inside the wall VW of the vessel, the latter position, which is referred to as subintimal. The tips 36, 42 of each device may be proximal or distal to the CTO. It is possible, as shown in FIGS. 6a, 6b and 6c, 6d, that one tip 36 or 42 is within the arterial occlusion or plaque and the other connection tip 42 or 36 is adjacent to it or within the subintimal space. Providing increased connection locations offers the medical practitioner flexibility in positioning each guide 32, 40 in order to make the connection.

During use of the device 30 in a recanalization procedure, the individual components 32, 40 of the device are inserted inside the body using the retrograde tracking (CART) technique or the reverse CART technique, shown in FIG. 1. Other techniques may also be used. Using such techniques, the guide wire 40 associated with the device and the catheter 32 associated with the device are each mounted with the connection tips 36, 42 having opposite polarity magnets 50 at the end of each respective tip to facilitate magnetic attraction of one tip to the other. The wire and catheter 32, 40 are guided using the desired techniques to positions within the vessel on either side of the occlusion CTO and into the occlusion CTO, as shown in FIG. 6b, or adjacent the occlusion CTO within the subintimal space, as shown in FIG. 6c, until the magnetic tip 36 is positioned within the closest proximity to the magnetic tip 42 of the catheter 40.

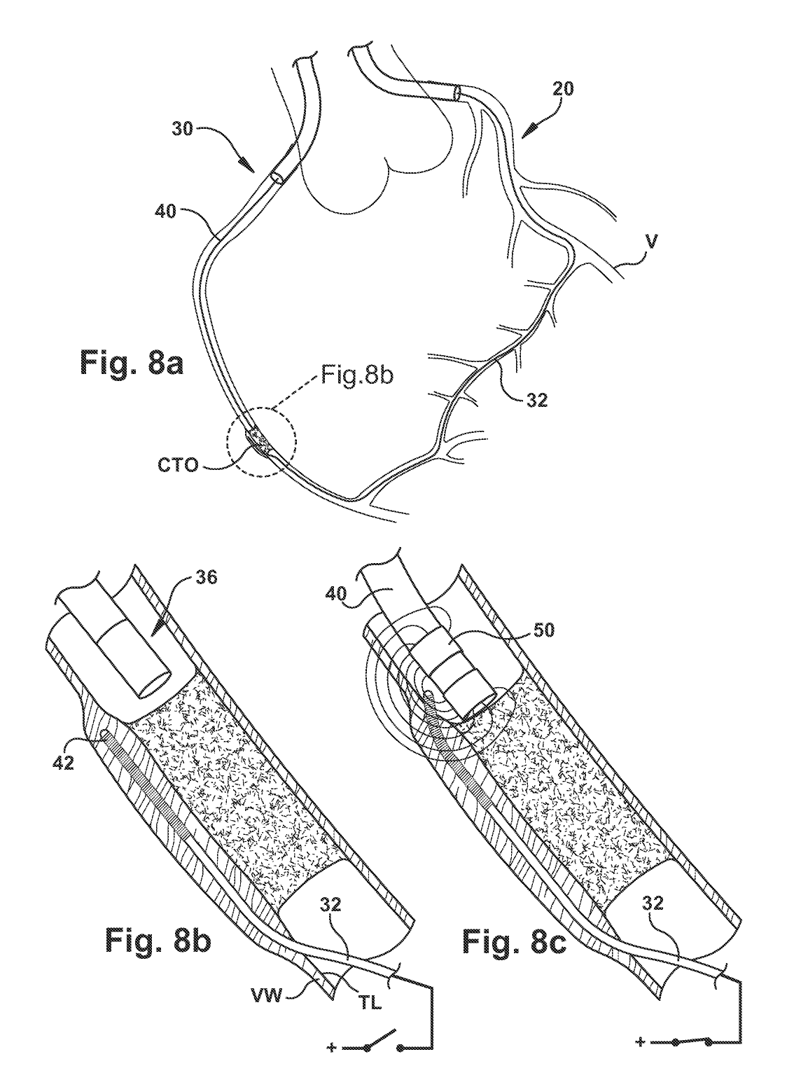

Using the magnetic pull of the opposite polarity of one, two or more rare earth magnets 50 in the tip 42 of the wire 40 to guide it toward the magnetic tip 36 of the catheter 32. Using the magnetic force, the tips 36, 42 of the devices 32, 40 will move into a desired aligned position, based upon placement of the magnets 50 on the respective instrument tips, and will form and lie in a straight line or a nearly connected straight line. Alternatives to magnetic guidance, such as an electromagnetic coil shown in FIGS. 8a, 8b and 8c, where the coil is embedded in either the first wire or the second catheter, may also be used. Upon activation of a coil, as shown by the open circuit in FIG. 8b which moves to closed and activated in FIG. 8c, where a magnetic attraction is shown as created between the other of either the second catheter or the first wire.

The intervening tissue separating the wire and the catheter may then be more easily breached by inserting into the occlusion a mechanical element 52, such as a hard tip wire, blade, needle or the like, as shown in FIG. 6a, or other such mechanical force techniques that are known to medical practitioners in this field.

Alternatively, as illustrated in the preferred embodiment of FIG. 6d, energy E such as in the form of electromagnetic force, radio frequency or a laser may be used to form a channel, with RF energy being preferred to breach tissue and create a connection. An external source for generating the energy force is capable of delivering penetrative energy from the magnetically sensitive tip of the first wire 32 to the magnetically sensitive tip of the second catheter 40 to open the occlusion. Using the tips 36, 42 in this aligned and close proximity position, means less energy and heat are required to be applied to form the channel. Reductions in energy and heat reduce the risks associated with damage to the vessel V.

Once the channel has been formed, a further wire W may then be threaded through the channel to move the wire into a central lumen of the catheter to a complete physical loop connection and substitute a wire for other devices such as balloon catheters, stents or other appropriate devices, which are passed over the wires to open the occlusion for revascularization.

In the illustrated embodiment of FIG. 7, the device 30 may further include an imaging probe IP provided on one or the other of the wire 32 or catheter 40 tips. In this embodiment, the device 30 is used across various chambers of the heart or across other cardiac structures. For example, the device 30 may be used to create a connection enabling medical practitioners to plug a hole, such as crossing atrial septal defects ASD, paravalvular leaks or ventricular septal defects. An imaging probe may be advantageous in such procedures, and may comprise an intra-vascular ultrasound catheter or an optical coherence tomography device.

During performance of a procedure using the device of this application to revascularize a vascular occlusion, the first guide 32 having a magnetic tip 36 and energy delivery or receiving portion 38 is moved via an antegrade path toward an occlusion CTO. Next, a second guide 40 having a magnetic tip 42 and energy receiving or delivery portion 44 is moved, via a retrograde path toward an opposite end of the occlusion CTO. There after, the first or second guide 32, 40 is engaged within a vessel wall VW or subintimal space adjacent the vascular occlusion CTO to be revascularized. Also, the other of the first or second guide, 40, 32, is engaged within the vascular occlusion CTO to be revascularized. The guides 32, 40 are positioned through magnetic attraction between their respective tips 36, 42, to minimize the space between the respective tips. Once the space is minimized, a minimal required energy is delivered from an energy delivery portion 38, 44 of either the first guide or the second guide 32, 40, and energy is received from the energy receiving portion 44, 38 of the other of either the first guide or the second guide 40, 32 to breach the vascular occlusion CTO and connect the magnetic tip 36 of the first guide 32 with the magnetic tip 42 of the second guide 40 through the opening created in the vascular occlusion to complete a pathway between the first and second guides.

It is understood that various alternative devices are available to medical practitioners for any desired use of the procedure, for example, either the first guide 32 is an antegrade delivered catheter and the second guide 40 is a retrograde delivered guide wire, or the first guide 32 is an antegrade delivered guide wire and the second guide 40 is a retrograde delivered guide wire, or the where first guide 32 is an antegrade delivered catheter and the second guide 40 is a retrograde delivered catheter.

Although the medical device of the present application has been described in detail sufficient for one of ordinary skill in the art to practice the invention, it should be understood that various changes, substitutions and alterations may be made without departing from the spirit or scope of the device as defined in the attached claims. Moreover, the scope of the present device is not intended to be limited to the specific embodiments described here, which are provided by way of example. As one of ordinary skill in the art will readily appreciate from the disclosure of the present device and its embodiments, other components and means presently existing or later to be developed that perform substantially the same function to achieve substantially the same result as those of the corresponding embodiments described here, may be utilized according to the present application. Accordingly, the appended claims are intended to include within their scope such other components or means.

We claim:

1. An improved medical device for revascularization of an occlusion comprising,
a first wire having a magnetic tip for engaging an occlusion or a subintimal space of a vessel adjacent an occlusion,
a second catheter having a central lumen and a magnetic tip, and
the first wire and second catheter magnetic tips each include a rare earth magnet, having opposite polarity, the magnetic force of the rare earth magnets being sufficiently strong to create a magnetic attraction sufficient to place the first wire and second catheter in close proximity to one another.

2. The improved medical device of claim 1 wherein the magnetic tip of each of the first wire tip and second catheter tip includes two or more rare earth magnets positioned to attract the rare earth magnets on the other tip.

3. A system for recanalization of an occlusion comprising:
a first wire with a magnetically sensitive tip which is placed in a vessel wall or subintimal space of a blood vessel,
a second catheter with a magnetically sensitive tip attracted to the magnetic sensitive tip of the first wire, which second catheter is placed on the other side of an occlusion in a vessel wall or subintimal space within the same blood vessel, and
an external source for generating energy force capable of delivering penetrative energy from the magnetically sensitive first wire to the second catheter to open the occlusion.

4. The system of claim 3, wherein the penetrative energy delivered from the external source for generating energy is radiofrequency energy.

5. The system of claim 3, wherein the external source for generating energy is a laser source.

6. The system of claim 3 wherein the catheter tip further includes an imaging probe comprising an intra-vascular ultrasound catheter.

7. The system of claim 3, wherein the catheter tip further includes an imaging probe consisting of an optical coherence tomography device.

8. The system of claim 3 wherein the magnetic sensitive tip includes an electromagnetic coil embedded in either the first wire or the second catheter, which coil is activated to create a magnetic attraction between the other of either the second catheter or the first wire.

9. The system of claim 3 wherein the penetrative energy delivered is mechanical energy.

10. The system of claim 3 wherein the occlusion to be recanalized is within a blood vessel of the heart.

11. The system of claim 3 wherein the occlusion to be recanalized is within a blood vessel of the leg.

12. The system of claim 3 wherein the occlusion to be recanalized is within a blood vessel within an organ of a human body.

13. The system of claim 3 wherein the occlusion to be recanalized is within a venous structure in a human body.

14. A method for revascularization of a vascular occlusion comprising the steps of:
moving a first guide having a magnetic tip and energy delivery or receiving portion via an antegrade path toward an occlusion;
moving a second guide having a magnetic tip and energy receiving or delivery portion via a retrograde path toward an opposite end of the occlusion;
engaging the first or second guide within a vessel wall or subintimal space adjacent the vascular occlusion to be revascularized;
engaging the other of the first or second guide within the vascular occlusion to be revascularized;
positioning the first guide and second guide, through magnetic attraction between their respective tips, to minimize the space between the respective tips;
delivering minimally required energy from the energy delivery portion of either the first guide or the second guide;
receiving energy from the energy receiving portion of the other of either the first guide or the second guide to breach the vascular occlusion;
connecting the magnetic tip of the first guide with the magnetic tip of the second guide through the opening created in the vascular occlusion to complete a pathway between the first and second guides.

15. The method of claim 14 wherein the first guide is an antegrade delivered catheter and the second guide is a retrograde delivered guide wire.

16. The method of claim 14 wherein the first guide is an antegrade delivered guide wire and the second guide is a retrograde delivered guide wire.

17. The method of claim 14 wherein the first guide is an antegrade delivered catheter and the second guide is a retrograde delivered catheter.
